(19) United States
(12) Patent Application Publication
(10) Pub. No.: US 2023/0371795 A1
(43) Pub. Date: Nov. 23, 2023

(54) SIDE VIEWING ENDOSCOPIC ENDCAP

(71) Applicant: Vanderbilt University, Nashville, TN (US)

(72) Inventors: Joshua GAFFORD, Nashville, TN (US); Robert J. WEBSTER, III, Nashville, TN (US); Patrick L. ANDERSON, Nashville, TN (US); Scott WEBSTER, Nashville, TN (US)

(21) Appl. No.: 18/030,434

(22) PCT Filed: Oct. 5, 2021
(86) PCT No.: PCT/US21/53539

§ 371 Date: Apr. 5, 2023

Related U.S. Application Data
(60) Provisional application No. 63/087,675, filed on Oct. 5, 2020.

(51) Int. Cl. A61B 1/00; A61B 1/018; A61B 1/015; A61B 1/12

(52) U.S. Cl. CPC A61B 1/00137; A61B 1/00177; A61B 1/018; A61B 1/00098; A61B 1/015; A61B 1/126; A61B 1/00085; A61B 1/00103; A61B 1/2736

(57) ABSTRACT

A system includes an endcap configured to be fitted on a distal end of an endoscope having a forward-looking camera and a forward-facing working port. The endcap includes a body configured to fit onto the distal end of the endoscope and a reflective surface supported by the body. The reflective surface is configured so that the forward-looking camera visualizes a workspace that is lateral of both the endcap and the distal end of the endoscope.

### RELATED APPLICATION

This application claims priority from U.S. Provisional Application No. 63/087,675, filed 5 Oct. 2020, the subject matter of which is incorporated herein by reference in its entirety.

### BACKGROUND

Bile ducts carry bile from the liver into the combined bile duct, which connects with the gallbladder and duodenum. Pancreatic ducts carry pancreatic juice from the pancreas to the duodenum, with small pancreatic ducts emptying into the main pancreatic duct. The common bile duct and main pancreatic duct join before emptying into the duodenum through the duodenal papilla.

Endoscopic retrograde cholangiopancreatography (ERCP) procedures are performed to diagnose and treat problems of the bile and pancreatic ducts. One condition for which ERCP can be performed is narrowing or blockage of the bile or pancreatic ducts. This condition can result from a variety of conditions, such as gallstones that form in the gallbladder and become stuck in the common bile duct, infection, pancreatitis, trauma or surgical complications in the bile or pancreatic ducts, pancreatic pseudocysts, and tumors/cancers of the bile ducts or pancreas.

The standard-of-care for performing ERCP is to use a duodenoscope, which is a specialized endoscope with a side-viewing camera and a deflection mechanism, referred to as an elevator, that makes it possible to position tools into the duodenal papilla. The doctor feeds the endoscope down the esophagus, through the stomach, and into the duodenum. A small camera mounted on the endoscope sends a video image to a monitor.

During ERCP, the doctor locates the opening where the bile and pancreatic ducts empty into the duodenum (the duodenal papilla) and uses the elevator mechanism to insert a delivery device, such as a catheter, through the endoscope and into the ducts via the papilla. Once the delivery device/catheter is positioned in the duct, the doctor can use it to perform various procedures, such as:
Inject a contrast medium into the ducts to make the ducts more visible on x-rays in order to enable the use of fluoroscopy to examine the ducts and look for narrowed areas or blockages.
Insert a tool configured to open blocked or narrowed ducts.
Insert a tool configured to break up or remove stones.
Insert a biopsy tool to perform a biopsy or remove tumors in the ducts.
Install stents in narrowed ducts to hold them open.

Duodenoscopes are reusable instruments. The inclusion of the elevator makes it extremely difficult to disinfect and sterilize the duodenoscope. This has led to alarmingly high rates of cross-contamination between cases. An FDA study in 2019 found that numerous recent ERCP deaths and infections over the past few years have been due to multi-drug-resistant bacteria transferred between patients undergoing ERCP. The study determined that the cause of the infections was that duodenoscopes—a specialized endoscope used to perform ERCP procedures—were transmitting bacteria to patients. The study found that bacteria, including Escherichia coli (E. coli), Staphylococcus, and carbapenem-resistant Enterobacteriaceae, was being transmitted between patients in 5.4% of all cases.

Furthermore, the FDA study linked the high infection rates directly to the elevator mechanism of the duodenoscope. The elevator mechanism allows the surgeon to perform ERCP procedures by allowing the surgeon to change the angle at which tools extend from the tip of the duodenoscope. To facilitate this function, the elevator includes several small moving parts. Small gaps that exist between the elevator parts harbor dangerous bacteria and are difficult to clean and sterilize between uses. Based on the infection rates found in the FDA study, approximately 40,000 patients every year are contracting duodenoscope-related infections.

These infections are not just the result of a failure to follow manufacturer cleaning guidelines. A U.S. Senate study found that contamination can happen even when all cleaning and sterilization processing steps are correctly followed. As a result, it is nearly impossible to predict when contamination will occur when using duodenoscopes with side elevator structures to perform ERCP procedures. These realizations led to the aforementioned FDA study, which emphasizes the urgent need to develop “innovative device designs, such as those with disposable components”, and urges hospitals to rapidly adopt these technologies, once available. The Senate report echoed these themes, calling for a concerted effort focused on curbing the “preventable tragedies” caused by these infections.

### SUMMARY

A disposable, side-viewing endcap makes it possible to perform endoscopic interventions at an angle with respect to the tip of an endoscope, such as a gastroscope, thus eliminating the need for a duodenoscope and the elevator mechanism associated therewith. The endcap contains a mirror that enables a forward-looking endoscope camera to provide a side view of an area of tissue, positioned laterally of the endoscope, which is normally outside the endoscope's view. The endcap allows a steerable sheath to be delivered through the endoscope and work within the side field of view afforded by the mirror. Utilizing the endcap with a forward looking endoscope, the steerable sheath can be positioned in the lateral workspace outside the endoscope within the side field of view afforded by the mirror. Surgical tools and other therapeutics can be delivered via the sheath.

An example where the capabilities facilitated by this endcap can be beneficial is in performance of ERCP procedures. The endcap can render the ERCP procedure capable of being performed with a standard, forward-looking flexible endoscope, such as a gastroscope, as opposed to specialized duodenoscopes which are the current standard of care. The steerable sheath re-directs and aims endoscopic tools at an angle, laterally or sideways from the tip of the endoscope, to facilitate the surgical procedure while the specialized mirror structure allows the surgeon to view the surgical site via the standard, forward-facing optics of the endoscope.

For example, in the ERCP procedure, the lateral tool redirection afforded by the endcap can facilitate cannulation of the duodenal papilla, with the mirror enabling lateral visualization of the surgical site at the duodenal papilla. When the endcap is coupled with a steerable sheath, the dexterity of the steerable sheath replaces the elevator function of a duodenoscope, which enables tool positioning for papilla cannulation. At the same time, the side-viewing mirror replaces the side-viewing camera in a duodenoscope, enabling visualization of the surgical site.

The combination of the side-view endcap and a steerable sheath provides a surgical system that enables standard forward-viewing gastroscopes to exhibit all of the functionality of specialized duodenoscopes, without any of the contamination concerns associated with the duodenoscope's elevator mechanism. The endcap is disposable and therefore discarded following the ERCP procedure, so all that is left is a standard gastroscope and the steerable sheath, both of which are capable of thorough cleaning and sterilization to repeatably and reliably avoid the threat of spreading infection through the harboring of bacteria. In fact, the steerable sheath can also be disposable, leaving only the endoscope to disinfect/sterilize.

In an example configuration, the endcap can be made from injection-molded biocompatible polycarbonate (PC), with an installed mirror that re-directs a portion of the endoscope field of view laterally in order to re-create the side-viewing capabilities of duodenoscopes. Because the cap contains no moving parts and can be a single piece of injection-molded plastic fitted with a laser-cut reflective mirror, it can be made very inexpensively while, at the same time, improving the sterilization characteristics of the surgical system.

Furthering the sterilization of the surgical system, the disposable, side-viewing endcap can be packaged in combination with a disposable steerable sheath. The sheath can be implemented as an add-on to a standard clinically-available forward-looking endoscope. In this instance, the addition of the sheath and endcap provides the function of a specialized duodenoscope.

According to one aspect, a system includes an endcap configured to be fitted on a distal end of an endoscope having a forward-looking camera and a forward-facing working port. The endcap includes a body configured to fit onto the distal end of the endoscope and a reflective surface supported by the body. The reflective surface is configured so that the forward-looking camera visualizes a workspace that is lateral of both the endcap and the distal end of the endoscope.

According to another aspect, the reflective surface can be positioned in an interior of the body, and the body can include a side opening through which the forward-looking camera visualizes the lateral workspace.

According to another aspect, the forward-looking camera can be directed parallel to a central axis of the endoscope. The reflective surface can be angled with respect to the central axis so that the forward-looking camera views the lateral workspace via a reflection of the lateral workspace on the reflective surface.

According to another aspect, the system can include a mirror that includes the reflective surface.

According to another aspect, the reflective surface can be convex and, thus, can increase the field-of-view (FOV) of the lateral workspace visualized by the camera.

According to another aspect, the convex reflective surface can be spherical.

According to another aspect, the mirror can have a length and a width, the length being greater than the width. The convex reflective surface can be aspherical. The radius of the convex reflective surface can be larger across the length of the mirror. The radius of the convex reflective surface can be smaller across the width of the mirror.

According to another aspect, the reflective surface can include a flattened central portion surrounded by spherical or aspherical edge portions.

According to another aspect, the system can also include a steerable sheath configured to extend through the working port and exit the distal end of the endoscope. The steerable sheath can have an inner lumen through which one or more tools can extend.

According to another aspect, the steerable sheath can include a flexible tube having a steerable tip. The tip can be actuatable to form a bend that enables the sheath to be steered outside the endcap in the lateral workspace.

According to another aspect, the endcap can include a redirecting surface configured to receive the steerable sheath and redirect the steerable sheath outside the endcap into the lateral workspace.

According to another aspect, the redirecting surface can be a portion of the reflective surface.

According to another aspect, the redirecting surface can be configured to enforce the lateral direction at which the steerable sheath exits the endcap into the lateral workspace.

According to another aspect, the steerable sheath can include a concentric tube structure including nested concentric tubes. According to this aspect, actuation of the steerable tip can be effectuated through applying an axial push-pull force to the concentric tubes.

According to another aspect, the concentric tubes can be configured to have asymmetric elasticity along the portions of the tubes that extend along the steerable tip.

According to another aspect, the asymmetric elasticity of the tubes can be created by cutout sections spaced lengthwise along the portions of the tubes that extend along the steerable tip.

According to another aspect, the tools can be tools for performing an endoscopic retrograde cholangiopancreatography (ERCP) procedure.

According to another aspect, the tools can include guidewires, cannulas, sphincterotomes, and baskets.

According to another aspect, the system can also include an endoscope having a forward-looking camera and a forward-facing working port. The endcap can be fitted onto the endoscope and the steerable sheath passes through the working port into the interior of the endcap. The endcap can be configured to deflect the steerable sheath toward a side opening of the endcap into the lateral workspace. The steerable sheath can be configured to be steered to a position and orientation in the lateral workspace, under the visualization afforded by the camera via the reflective surface, to allow a tool to be advanced through the steerable sheath into the lateral workspace.

According to another aspect, the endoscope can include an illumination source and the reflective surface is configured to reflect light from the illumination source into the lateral workspace to illuminate the lateral workspace.

According to another aspect, the endoscope can include a fluid delivery channel and the endcap can be configured so that fluids delivered via the fluid delivery channel wash the reflective surface.

According to another aspect, the endcap body can include a single piece of injection molded plastic configured to receive and support the reflective surface.

According to another aspect, the endcap body can be free from moving parts.

According to another aspect, the endcap body can include a collar configured to be press-fitted onto the distal end of the endoscope tube.

According to another aspect, the body of the endcap can include a tip portion including flexible members configured to deflect in response to engaging tissue in order to facilitate navigation of the endoscope with the tip affixed thereto.

According to another aspect, the endcap can also include a tip including a separate component connectable with the body. The tip can have a domed configuration and can be constructed of a material that is soft and flexible. The tip can be configured to deflect in response to engaging tissue in order to facilitate navigation of the endoscope with the tip affixed thereto.

According to another aspect, the endcap can also include a basket. The basket can include a plurality of loops and/or fingers that extend radially from the endcap body. The loops and/or fingers can be constructed of a material that is flexible and resilient so that the loops deflect toward the endcap body while the endoscope is advanced through tissue. The loops and/or fingers can be configured to engage tissue and to deflect outward of the endcap body to space tissue from the endcap and to support the endcap and distal end of the endoscope in the workspace.

According to another aspect, an endcap configured to be fitted on a distal end of an endoscope having a forward-looking camera and a forward-facing working port can include a body configured to fit onto the distal end of the endoscope. The body can include a side opening providing access from an interior of the endcap to a lateral workspace exterior of the endcap. The workspace can be lateral to both the endcap and the distal end of the endoscope. The endcap can also include a mirror supported in the interior of the endcap. The mirror can include a reflective mirror surface in the field-of-view (FOV) of the forward-facing camera. The mirror surface can be configured to place the lateral workspace within the FOV of the forward facing camera.

### BRIEF DESCRIPTION OF THE DRAWINGS

The foregoing and other features and advantages of the present disclosure will become apparent to one skilled in the art to which the present disclosure relates upon consideration of the following description of the invention with reference to the accompanying drawings, wherein like reference numerals, unless otherwise described refer to like parts throughout the drawings and in which:

FIG. 1 is an exploded perspective view of an example configuration of a system including an endcap configured to be fitted on a distal end of an endoscope.

FIG. 2 is a perspective view of the system.

FIGS. 3A and 3B illustrate an example implementation of the system.

FIG. 4A is a perspective view of an example configuration of a steerable sheath that can form a portion of the system.

FIGS. 4B and 4C are sectional views illustrating the operation of the steerable sheath of FIG. 4A.

FIGS. 5 and 6 are perspective views of an example configuration of an endcap that can form a portion of the system.

FIG. 7 is an exploded view of the endcap of FIGS. 5 and 6.

FIGS. 8A-8C illustrate a first example configuration of a mirror that can be implemented in the endcap.

FIGS. 9A-9C illustrate a second example configuration of a mirror that can be implemented in the endcap.

FIGS. 10 and 11 are perspective views of another example configuration of an endcap that can form a portion of the system.

FIGS. 12A and 12B are elevation views illustrating different conditions of the endcap of FIGS. 10 and 11.

FIG. 13 is an exploded view of the endcap of FIGS. 10 and 11.

### DETAILED DESCRIPTION

### The Surgical System

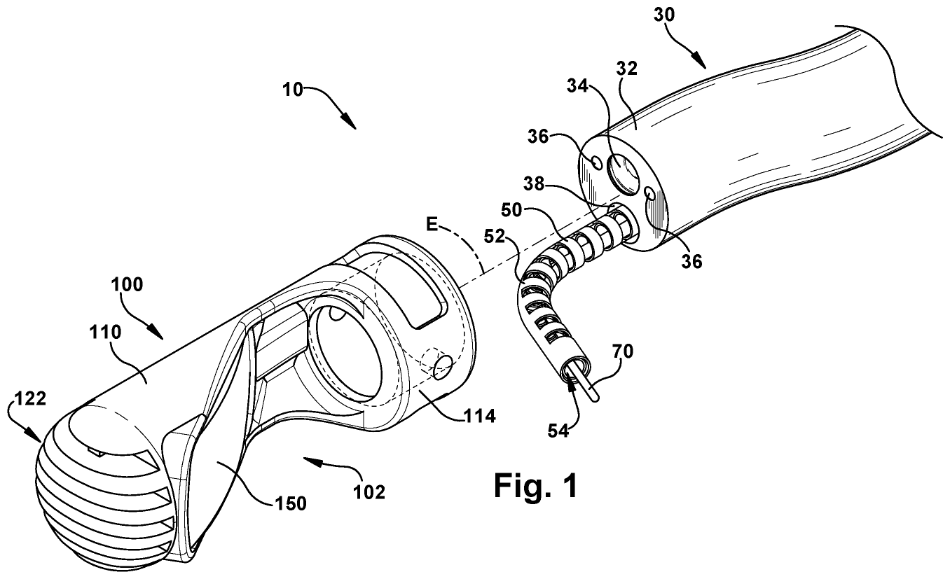

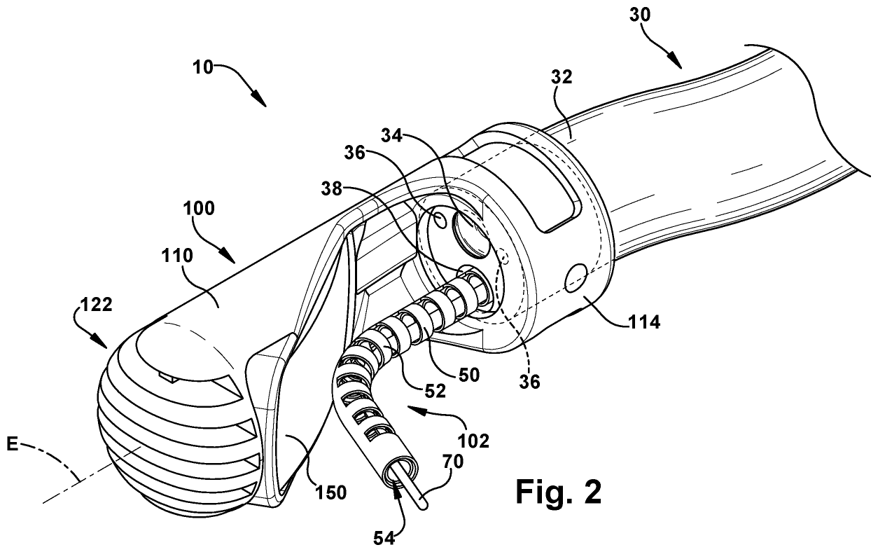

FIGS. 1 and 2 illustrates an example configuration of a surgical system 10 for performing endoscopic surgery. The system 10 includes an endoscope 30, a surgical tool 70 delivered by the endoscope, and an endcap 100 for facilitating a side view from the tip 32 of the endoscope. The endcap 100 is suitable for providing a side view for any endoscopic surgical procedure in which a side view might be beneficial to the surgeon. In the example configuration described herein, the endoscope 30 is a flexible gastro-intestinal (GI) endoscope or gastroscope, with the endcap 100 affixed to its distal end or tip 32 to configure the gastroscope to act as a duodenoscope. Configured as such, the endcap 100 enables the system 10 to be used to perform an endoscopic retrograde cholangiopancreatography (ERCP) procedure.

The endoscope 30 includes a camera 34 and illumination sources 36 (e.g., fiber optics or LEDs) for providing visualization of the workspace in the area of the endoscope tip 32. The endoscope 30 also includes at least one inner lumen or working port 38 through which surgical tools can be guided. The endoscope 30 can include other features (not shown), such as ports for introducing air to expand the workspace and/or irrigation fluids (e.g., saline solution) for clearing/cleaning the workspace and/or the endcap 100. The camera 34, illumination sources 36, and working port 38 are all positioned on an axial end face of the endoscope 30 and face parallel to the endoscope axis E.

The endoscope 30 can be of a conventional, commercially available, off-the-shelf flexible endoscope/gastroscope configuration. GI endoscopes/gastroscopes can have diameters that range from 9 mm to 14 mm, with working port 38 diameters in the range of 2.7 mm-3.8 mm. Examples of these commercially available forward-looking endoscopes/gastroscopes include the Olympus® EXERA PCF-160AL model, which was used to test the efficacy of the endcap 100.

The surgical system 10 also includes a steerable sheath 50 that is introduced through the working port 38 of the endoscope 30. The steerable sheath 50 has an end portion 52 that is actuatable to take on a curvature which allows the end portion to be steered to a desired position and orientation outside the endcap 100. The surgical tool 70 can be passed through the sheath 50 to access the surgical site. The sheath 50 is steered remotely from a proximal end of the endoscope 30 via an external controller (not shown). The controller can be mechanical for manual operation of the sheath, or robotic for computer-controlled operation, either automatically or in response to user control inputs.

### ERCP Procedure

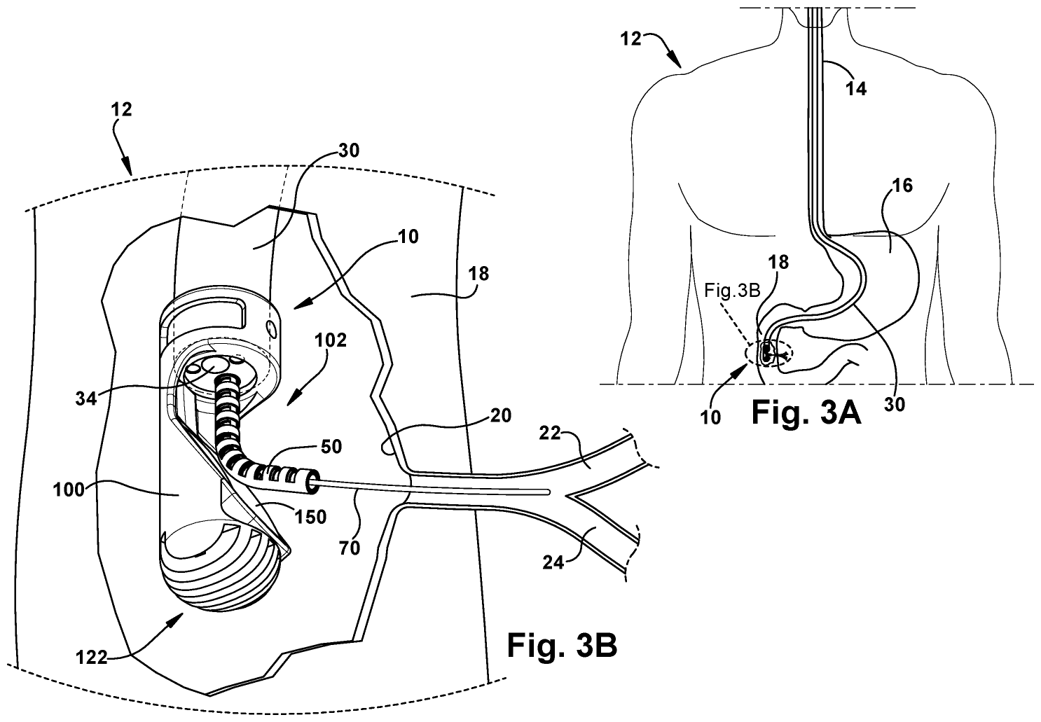

FIGS. 3A and 3B illustrate the surgical system 10 performing an ERCP procedure on a subject 12. As shown in FIG. 3A, the endoscope 30 is delivered through the subject's esophagus 14 and stomach 16 to the duodenum 18. As shown in FIG. 3B, the flexible endoscope 30 is maneuvered to position a side opening 102 of the endcap 100 facing the major duodenal papilla 20, where the common bile duct 22 and the main pancreatic duct 24 meet and enter the duodenum 18. The endcap 100 facilitates maneuvering the steerable sheath 50 through the side opening 102 toward the duodenal papilla 20 so that the surgical tool 70 can be maneuvered through the duodenal papilla to treat the common bile duct 22 and/or the main pancreatic duct 24.

### Steerable Sheath

The configuration of the steerable sheath 50 is important as it lends to the advantageous efficacy of the surgical system 10. As noted above, the standard gastroscope 30 upon which the endcap 100 is fitted, and through which the steerable sheath 50 is passed, has a working port 38 diameter ranging from 2.7 mm-3.8 mm. The outside diameter (OD) of the steerable sheath 50 therefore must be configured to fit through the working port 38 while, at the same time, be configured to accommodate the passage of the tool 70 through its inside diameter (ID). The steerable sheath 50 therefore needs to avoid structure along its length, and especially at its distal end, that could interfere with the passage of the tool 70 through its ID, while its OD presents an acceptable fit within the ID of the working port 38.

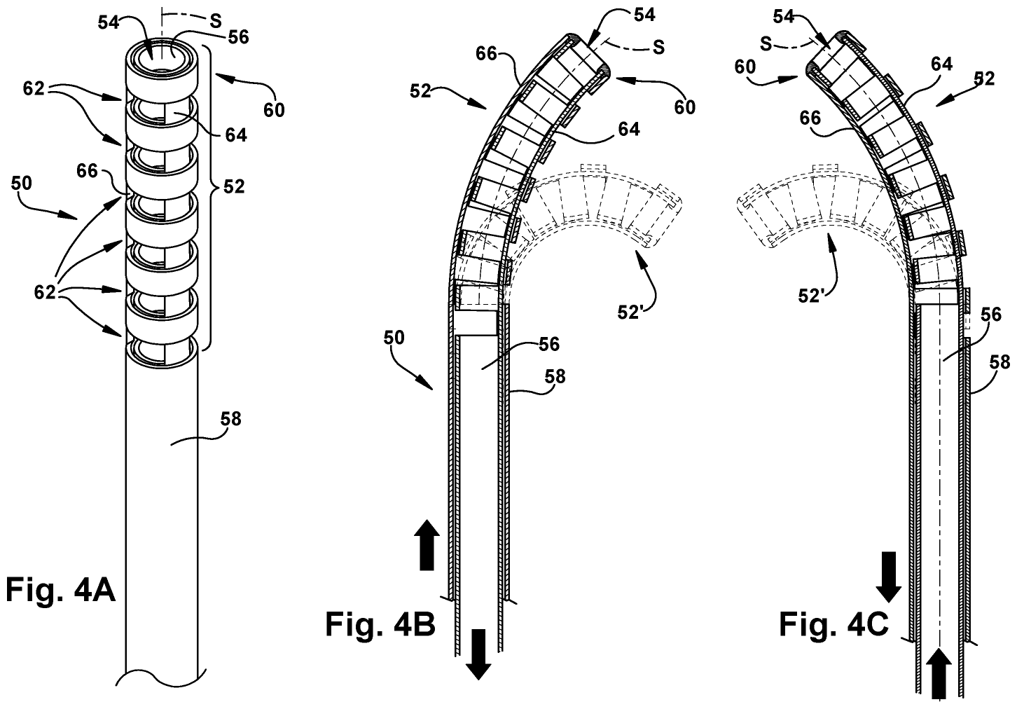

An example configuration of the steerable sheath 50 is illustrated in detail in FIGS. 4A-4C. The sheath 50 implements an asymmetrical stiffness approach to create bending in the end portion 52. The sheath 50 has an extremely thin-walled tubular configuration in which an inner lumen 54 of the sheath is free from obstruction by traditional bending mechanisms, such as pull-wires and hinges. These traditional bending mechanisms are far too large to fit through the working port 38 of the endoscope 30 while maintaining the ability to carry standard trans-endoscopic tools and instruments through the inner lumen 54.

The sheath 50 can, for example, have a concentric tube configuration similar or identical to any of those disclosed in U.S. patent application Ser. No. 15/804,146, filed on Nov. 6, 2017 (U.S. Patent Application Publication US 2019/0133705 A1) for SURGICAL DEVICE TIP WITH DEFLECTABLE JOINT. The disclosure of the '146 application is hereby incorporated by reference in its entirety.

As shown in FIGS. 4A-4C, the sheath 50 includes an inner tube 56 and an outer tube 58, which are connected to each other (e.g., via welding or adhesive bonding) at the tip 60 of the sheath 50. According to one example configuration, the tubes 56, 58 can be constructed of a shape memory alloy, such as a nickel-titanium alloy (“nitinol”). Nitinol allows for an extremely thin-walled construction of the tubes 56, 58, while the shape memory properties of the material causes the tubes to return to their pre-configured state once bending forces are removed. Other materials, such as surgical stainless steels or plastics/polymers, can also be used to construct the sheath 50. According to another example configuration, the tubes 56, 58 can be formed from laser-cut hypodermic tubes or “hypotubes.”

In the example configuration of the sheath 50, the pre-configured state of the sheath is the straight tubular configuration shown in FIG. 4A. The sheath 50 is configured to be actuatable to bend the end portion 52 in opposite directions, as shown in FIGS. 4B and 4C. To facilitate this function, each tube 56, 58 has material removed, e.g., via laser cutting, to form a slit pattern in the tubular sidewall along the end portion 52 of the sheath 50. In the example configuration of FIGS. 4A-4C, the slit pattern of the tubes 56, 58 is formed with rectangular notches 62 removed from the sidewalls of the tubes. The notches 62 leave intact a spline 64 of the inner tube 56 and a spline 66 of the outer tube 58.

The notches 62, and the resulting splines 64, 66, create an asymmetrical elasticity in the tubes 56, 58. Owing to this configuration, the tip portions of the tubes 56, 58 are axially stiff along the splines 64, 66 and axially compliant along the notches 62. This asymmetrical elasticity makes the tip portions 52 of the tubes 56, 58 easily bendable along their respective splines 64, 66.

In the example configuration illustrated in FIGS. 4A-4C, the notches 62 in the tubes 56, 58 face in radially opposite directions with respect to the sheath axis S. Because the tubes 56, 58 are connected at the tip 60, the sheath 50 can be actuated to cause bending of the tip portion 52 through exerting an axial push/pull force on the tubes 56, 58. In the illustrated configuration, due to the opposite facing configuration of the notches 62, the tip portion 52 is configured to bend in opposite directions. The direction in which the tip portion 52 bends depends on the push/pull directions of the actuation force exerted on the tubes 56, 58. This is shown in FIGS. 4B and 4C.

Referring to FIGS. 4B and 4C, the push/pull directions along which the actuation force is applied are described with reference to the user, i.e., the surgeon. Actuation force applied in the “push” direction is therefore applied along the sheath axis S away from the surgeon, i.e., toward the tip 60. Actuation force applied in the “pull” direction is therefore applied along the sheath axis S toward from the surgeon, i.e., away from the tip 60. Following this convention, in FIG. 4B, a pull force is applied to the inner tube 56 and a push force is applied to the outer tube 58. Similarly, in FIG. 4C, a push force is applied to the inner tube 56 and a pull force is applied to the outer tube 58.

The push/pull force applied to the tubes 56, 58 is relative and, therefore, can be realized through the application of axial force on one tube only. Therefore, the actuation forces applied to the tubes 56, 58 as identified in FIGS. 4B and 4C can be realized through one or both of the identified push/pull force applications. In other words, in FIG. 4B, the actuation force can be realized by applying a pulling axial force to the inner tube 56 while maintaining outer tube 58 in a fixed axial position, by applying a pushing axial force to the outer tube 58 while maintaining the inner tube 56 in a fixed axial position, or by applying a pulling axial force on the inner tube 56 and simultaneously applying a pushing axial force on the outer tube 58. The corollary holds true for the bending motion shown in FIG. 4C.

When the actuation force is applied to the sheath 50, the relative axial force acts at the tip 60 where the tubes 56, 58 are interconnected. The actuation force is transmitted to the tip 60 via the splines 64, 66 and is therefore applied asymmetrically, i.e., offset from the sheath axis S. As a result, the tip portion 52 bends inward toward the axis S along the spine of the “pushing” tube. In FIG. 4B, the outer tube 58 is the pushing tube and the inner tube 56 is the pulling tube, so the end portion 52 bends along the outer tube spline 66—to the right as shown in FIG. 4B. In FIG. 4C, the outer tube 58 is the pulling tube and the inner tube 56 is the pushing tube, so the end portion 52 bends along the inner tube spline 64—to the left as shown in FIG. 4C.

The construction of the sheath 50, which requires only the tubes 56, 58 without any further hardware or mechanisms. This allows the sheath 50 to have a thin-walled construction while providing the ability to actuate or steer the end portion 52. This allows the sheath 50 to be compatible with both standard endoscopy equipment and standard trans-endoscopic tools, such as guidewires, cannulas, sphincterotomes, and baskets. In one example configuration, the sheath 50 can have a total wall thickness, i.e., the stacked thicknesses of the walls of both tubes 56, 58, of 0.15 mm, which is just slightly larger than the thickness of a human hair. This wall thickness is impossible to achieve with pull-wire-based systems, as the wires themselves are typically 0.15 mm or larger. The overall diameter of the sheath 50 can, for example, be 1-3.5 millimeters, and is therefore capable of passing easily through the working port 38 of the endoscope 30 (2.7-3.5 mm diameter) while accommodating typical ERCP tools.

Advantageously, the configuration of the steerable sheath 50 allows it to be small enough to fit through the working port 38 of the endoscope, with an internal diameter large enough to carry standard tools through the lumen 54, all while being steerable so the tools can be aimed without requiring the elevator mechanism used in conventional duodenoscopes. The ability to control the curved configuration of the end portion 52 allows it to exit the endcap 100 laterally and to position the tip 60 of the sheath 50 at a desired location and orientation in the workspace, laterally of the endoscope 30, so that the tool 70 can exit along the desired path or trajectory.

### Endcap Configuration

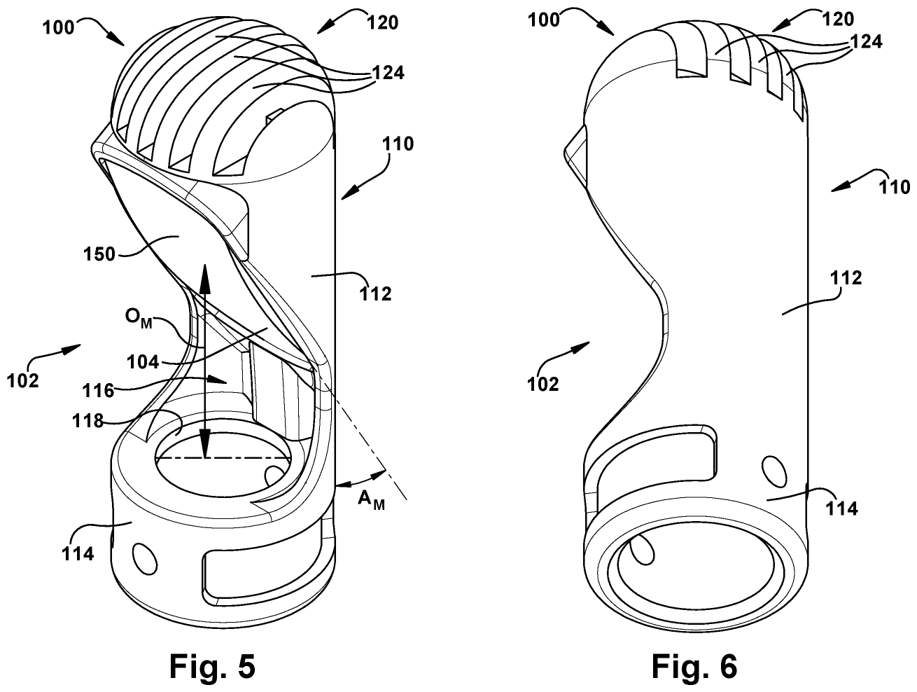

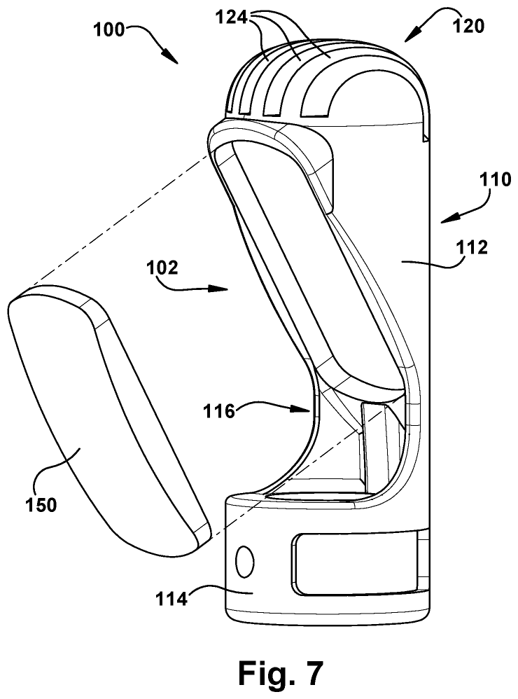

The steerable sheath 50 implemented in the surgical system 10 extends through the working port 38 of the endoscope 30. The sheath 50 is configured to exit the endcap 100 through the side opening 102, positioning it and its tip 60 in the workspace, which is lateral of both the endoscope 30 and the endcap. The endcap 100 is configured to facilitate viewing the lateral workspace, illuminated by the LEDs 36, with the axially facing camera 34 camera. To do this, the endcap 100 includes a side-view mirror 150 positioned oblique to the endoscope axis E in the view of the camera 34. Through the mirror 150, the camera can visualize the workspace laterally of the endcap 100 and the endoscope 30. One example configuration of the endcap 100 is illustrated in FIGS. 5-7.

Referring to FIGS. 5-7, the endcap 100 has a body portion 110 with a generally cylindrical sidewall 112. A portion of the sidewall 112 is removed to form the side opening 102. The side opening 102 provides access to an interior 116 of the endcap 100, where the mirror 150 is supported and into which the sheath 50 is directed from the endoscope 30.

A base portion 114 of the endcap 100 forms a collar configured to fit onto the tip 32 of the endoscope 30 and is sized to be secured thereto via an interference fit or press-fit/snap fit. For example, the base portion/collar 114 can be formed of a material, such as silicone rubber or thermoplastic elastomer, selected so that the collar is somewhat flexible or elastic to enable the press-fit and accommodate variations in the OD of the endoscope 30. An annular rim 118 of the base portion/collar 114 serves as an end stop for the tip 32 of the endoscope 30. Endcap 100 is configured such that the engagement between the tip 32 of the endoscope 30 and the annular rim 118 ensures a proper fit and alignment of the endcap 100 in a repeatable and reliable manner.

The endcap 100 includes an end portion 120. When the endcap 100 is installed on the endoscope 30, the end portion 120 forms a leading tip 122 of the assemblage. The end portion 120, serving as a leading tip 122, therefore can be tasked with engaging and navigating tissue as the endoscope 30 is delivered to the desired workspace. Accordingly, the end portion 120 can be configured to facilitate this function. One manner in which this can be achieved is through applying a domed configuration to the end portion 120. In the example configuration of FIGS. 5-7, the domed end portion 120 has a slotted configuration in which a series of fins 124 are formed. The fins 124 can lend some flexibility to the end portion 120, especially when constructed of a polycarbonate material which, if solid instead of slotted, would not deflect.

Alternatively, the domed end portion 120 can be free from fins and have a uniform, smooth, continuous surface (see, e.g. FIGS. 11-14). In either instance, the end portion 120, with or without fins, can be an integral portion of the endcap 100 or a separate component fitted onto the body 110 of the endcap. This can allow for forming the end portion 120 from a softer biocompatible material, such as soft (durometer 20-80 (Shore A)) silicone rubber or thermoplastic elastomer, in order to improve its navigability and reduce the possibility of tissue damage during delivery.

To facilitate the sheath's lateral exit through the side opening 102, the endcap 100 can include a deflecting surface 104 that is angled or otherwise curved and configured to receive the sheath 50 as it exits the working channel 38. The endcap 100 is configured so that the sheath 50 engages the deflecting surface 104 as it exits the working channel 38 of the endoscope 30. The deflecting surface 104 redirects the sheath 50 toward the side opening 102. In the example configuration illustrated in FIGS. 5-7, the deflecting surface 104 is a lower surface of the mirror 150. Alternatively, the length of the mirror 150 could be reduced and the deflecting surface 104 could be formed as a part of the molded endcap material. This could allow, for example, the deflecting surface 104 to have a curved or channeled configuration.

The redirection of the sheath 50, along with its steerable capabilities, facilitate positioning the end portion 52 and tip 60 of the sheath at a desired position and orientation in the workspace in order to provide tool access. The redirection effectuated by the deflecting surface 104 provides general or coarse control of the trajectory and path the sheath 50 takes toward and through the side opening 102. The actuatable steering features of the sheath 50 provides fine control of its trajectory and path. Acting in concert, the redirection afforded by the deflecting surface 104 of the endcap 100 and the steerable quality of the sheath 50, yields a high degree of tip 60 and end portion 52 control in the lateral workspace.

The endcap 100 directs the sheath 50 through the side opening 102 into the workspace under the visualization via the camera 34 afforded by the mirror 150. Under this visualization, the surgeon can actuate the sheath 50 to control its position and orientation to allow the surgical tool 70 to cannulate the duodenal papilla and/or aim the surgical tool in order to enable the ERCP procedure. Outfitted with the sheath 50 and the endcap 100, the conventional endoscope/gastroscope 30 can perform all of the functions provided by a duodenoscope while, at the same time, eliminating the elevator structure of the duodenoscope and the infection risks associated therewith.

The construction of the endcap 100 is configured to be simple and inexpensive to produce so that it can be a disposable, single-use product. To facilitate this, the endcap 100 can be constructed of a material or materials selected to achieve this purpose. For example, the endcap 100 can be constructed of a polymer material, such as injection molded biocompatible polycarbonate (PC), which is relatively inexpensive, thus allowing the endcap to possess the disposable, single-use configuration. This can be the case, for example, where the endcap 100 is configured to fit onto a specific endoscope configuration, where the dimensions are fixed.

As another example, the endcap 100 can be constructed of multiple materials. This can be the case, for example, where the endcap 100 is configured to fit onto a range of endoscope configurations/diameters or where certain performance characteristics are desired. For instance, portions of the endcap 100 where structural stability is desired can be constructed using a hard/strong material, such as a molded polycarbonate material. In this instance, portions of the endcap where softness and/or flexibility is desired, such as at the base portion/collar 114 or the tip/end portion 120, can be constructed using a soft/flexible material, such as a thermoplastic elastomer or silicone rubber material.

The endcap 100 is configured to receive secure the mirror 150. The connection between the endcap 100 and the mirror 150 can, for example, be through a press-fit connection, snap-in connection, or adhesive connection. The mirror 150 is a convex mirror configured to provide an optimal field of view FOV for the surgeon via the camera 34. The mirror 150 has several features or properties that are optimized to produce the optimal FOV.

### The Mirror

The mirror 150 can be molded from a rigid, hard material capable of providing a smooth base upon which to form the reflective mirrored surface. The mirror 150 can, for example have a molded biocompatible polycarbonate construction with a mirror surface 152 formed by an optical grade silver mirror coating and a protective overcoat. The mirror 150 has a generally rectangular shape or footprint, as shown in the figures. The reflective surface 152 can have various configurations selected to produce a clear reflected image and a wide field-of-view (FOV). According to one example configuration, the mirror 150, particularly the reflective surface 152, can have a convex configuration.

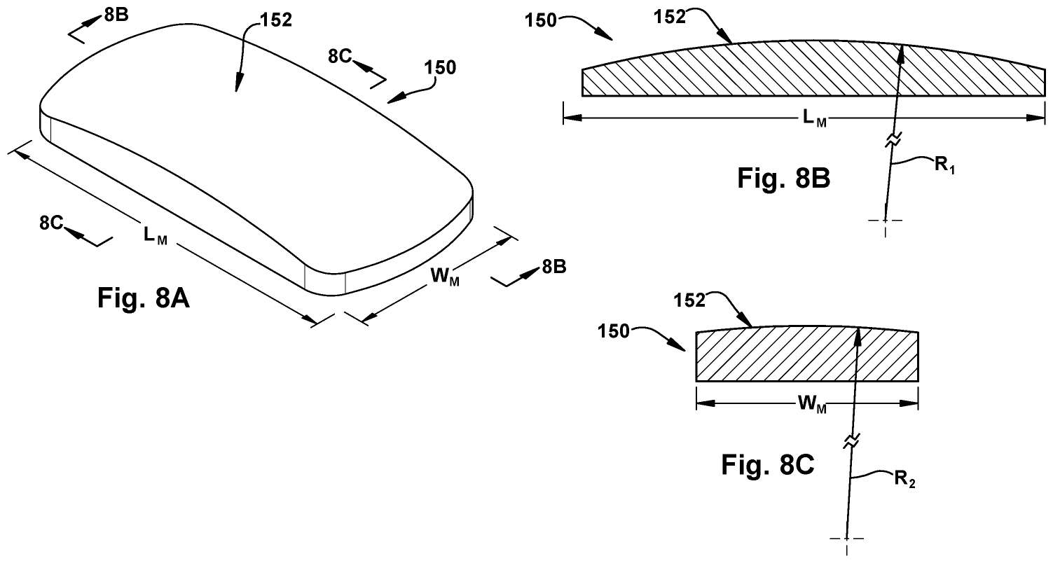

Referring to FIGS. 8A-8C, the mirror 150 has a length L_{M} and a width W_{M}. The mirror 150 of FIGS. 8A-8C is a convex mirror in which the reflective surface 152 is convexly curved in the length L_{M} dimension along a radius R₁ and in the width W_{M} dimension along a radius R₂. Since maximizing the FOV for the mirror is a goal for the endcap design the rectangular dimensions of the mirror 150 can be maximized within the confines of the interior 116 of the endcap, which are, in turn, defined by the size (i.e., diameter) of the endoscope 30.

The FOV of a convex mirror increases as the convex curvature of the mirror increases, i.e., the radius of the mirror surface is reduced. The image quality (sharpness, clarity, focus, etc.), however, degrades as the convex curvature increases/radius decreases. Any convex mirror will produce some distortion throughout the image, with the degree of distortion increasing at the edges of the mirror. Therefore, the greater the FOV due to an increased curvature/decreased radius of the mirror surface 152, the greater the degree of image distortion, especially at the edges. Since, however, the surgeon will instinctively position the sheath 50 and tool 70 in the middle of the FOV, it will be appreciated that some edge distortion can be acceptable.

Furthermore, for the implementation of the convex mirror 150 in the endcap 100, the mirror angle A_{M} with respect to the endoscope axis E and the mirror offset O_{M}, i.e., the distance between the mirror 150 and the camera 34 (measured in the direction of the endoscope axis E from the center of the camera lens), also affect the FOV, image quality, and edge distortion (see FIG. 5). Therefore, all of these factors—mirror surface curvature (R₁, R₂) mirror angle (A_{M}), and mirror offset (O_{M}), are taken into account when configuring the endcap 100 to provide an optimized image of the workspace.

According to one example configuration, the convex reflective surface 152 can be a spherical surface and, therefore, R₁=R₂. For a spherical mirror surface 152, noting that the mirror 150 is rectangular, it follows that the arclength of the mirror surface is shorter along the width W_{M} of the mirror than it is along the length L_{M}. It will therefore be appreciated that, for a spherical configuration of the convex mirror surface 152, the FOV in the length L_{M} dimension of the mirror 150 is wider than the FOV in the in the width W_{M} of the mirror. It may, however, be desirable for the FOV in the width W_{M} dimension of the mirror 150 that is on par with the FOV in the length L_{M} dimension of the mirror.

To achieve this, according to another example configuration, the mirror surface 152 can have an aspherical convex configuration in which R₂<R₁. The degree to which the radius R₂ is less than R₁ can be selected to widen the FOV in the width W_{M} dimension of the mirror 150 so that it is greater than that of a spherical mirror surface. The radius R₂ can, for example be reduced so that the FOV in both dimensions, length L_{M} and width W_{M}, are equal or on par with each other.

For either of the aforementioned example configurations, the parameters that affect the FOV, image quality, and distortion can be optimized to provide an ideal endcap function. The parameters of the endcap 100 and mirror 150 that can be adjusted to affect the image viewed via the camera 34 are the mirror surface parameters (R₁, R₂), the mirror angle A_{M}, and the mirror offset O_{M}.

Optimization can be performed in a variety of manners, ranging from trial-and-error to computer modeling and even artificial intelligence (AI). According to one example optimization method, the parameters R₁/R₂, A_{M}, and O_{M} are inputted into a raycasting model that computes a lateral visualization score. A Bayesian optimizer is used to tune the parameters to improve visualization, and those tuned parameters are fed back to the raycasting model to recalculate the visualization score. The process loops or repeats until optimized parameters are determined.

Of course, the optimized parameters cannot be determined without identifying an ideal FOV and acceptable levels of image distortion. Commercially available duodenoscopes have FOVs in the range of about 100 degrees. Examples of commercially available duodenoscopes include the Boston Scientific® Exalt model, the Olympus® EVIS-EXERA model, and the PENTAX® ED34-i10T2 model. Therefore, in one iteration of the optimization model, a goal of matching the 100 degree FOV of commercially available duodenoscopes was implemented.

Image distortion can be difficult to quantify, especially in regard to an image reflected from a convex mirror, where the distortion increases from center to edge. One manner in which distortion can be quantified is by measuring edge drop-off. Edge drop-off is the perceived spatial contraction (and reduction in detail) of the reflected image towards the edges of the mirror. For purposes of evaluating a particular configuration of the mirror 150, edge drop-off was quantified experimentally through a calibration process by reflecting a checkerboard pattern in the mirror and calculating how much ‘smaller’ the squares are at the edge of the mirror vs. at the geometric center. This center-to-edge differential in the checkerboard pattern was used as a drop-off parameter for which the performance of various mirror configurations was evaluated.

Finally, for a given mirror angle A_{M} and radius R₁/R₂, the mirror offset O_{M} will affect the FOV and edge drop-off for a given mirror configuration. For example, as the as the mirror offset O_{M} decreases, i.e., the mirror 150 moves closer to the camera 34, the FOV and edge drop-off are reduced. Conversely, as the as the mirror offset O_{M} increases, i.e., the mirror 150 moves away from the camera 34, the FOV and edge drop-off are increased. Of course, the mirror offset O_{M} is limited, both by the dimensions of the endcap 100 and its interior 116, and also by the fact that a portion of the mirror surface 152 can also act as the deflecting surface 104, so it needs to be positioned within a certain proximity of the working channel 38.

The optimized endcap parameters for an endcap 150 with a spherical mirror surface 152 were determined to fall within the following ranges:
Mirror Curvature (R₁=R₂): 25-100 mm.
Mirror Angle (A_{M}): 45-60 degrees.
Mirror Offset (O_{M}): 5-12 mm.

Within these ranges, an optimized set of endcap parameters for an endcap 100 including a mirror 150 with a spherical mirror surface 152 was determined to be a mirror angle A_{M} of 51 degrees, a mirror radius R₁=R₂ of 50 mm, and a mirror offset O_{M} of 10 mm.

The optimized endcap parameters for an endcap 150 with an aspherical mirror surface 152 were determined to fall within the following ranges:
Mirror Length (L_{M}) Curvature (R₁): 25-100 mm.
Mirror Width (W_{M}) Curvature (R₂): 10-50 mm.
Mirror Angle (A_{M}): 45-60 degrees.
Mirror Offset (O_{M}): 5-12 mm.

Within these ranges, an optimized set of endcap parameters for an endcap 100 including a mirror 150 with a spherical mirror surface 152 was determined to be a mirror angle A_{M} of 51 degrees, a mirror radius R₁ of 50 mm, a mirror radius R₂ of 25 mm, and a mirror offset O_{M} of 10 mm.

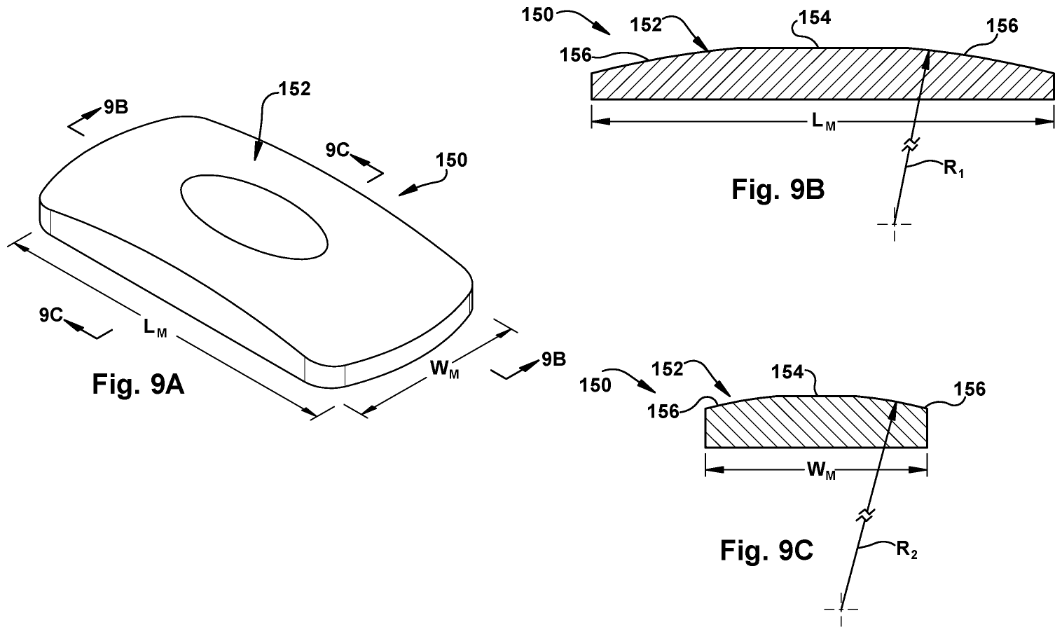

Another example configuration for the mirror 150 is illustrated in FIGS. 9A-9C. The mirror 150 is similar to the mirror of FIGS. 8A-8C, with the exception that the mirror surface 152 includes a flat central portion 154 and a convex peripheral portion 156. In this configuration, the central portion 154 provides the center of the FOV for the mirror 150. Because the central portion 154 is flat, the distortion associated with a curved mirror surface is eliminated. Because the surgeon will naturally or instinctively position the sheath 50 and/or the tool 70 centrally on the mirror 150, the image quality at the focal point of the workspace will be maximized. At the same time, the peripheral portion 156, being convexly curved as described above in regard to the mirror of FIGS. 8A-8C, will widen the FOV according to the mirror and endcap parameters.

In the example configuration illustrated in FIGS. 9A-9C, the central portion 154 has a generally elliptical configuration. The dimensions of the elliptical central portion 154 could be adjusted, for example, based on the type of procedure being performed, the anatomical structure in which the endoscope 30 and endcap 100 are being used, and user feedback/preferences.

For any of the endcap configurations disclosed herein, it is possible that biological debris could occlude or foul the mirror. To account for this, the endcap can be configured to clean the mirror 150 with the built-in water nozzle capabilities of the standard endoscope 30, which is already in-place and used to clean the endoscope camera 34. In one alternative construction, the endcap 100 can be configured to include an integrated irrigation channel connected to an external cleaning solution source, such as a luer lock syringe filled with saline. The cleaning fluid can be delivered via a thin polyurethane tube.

### Basketed Endcap Configuration

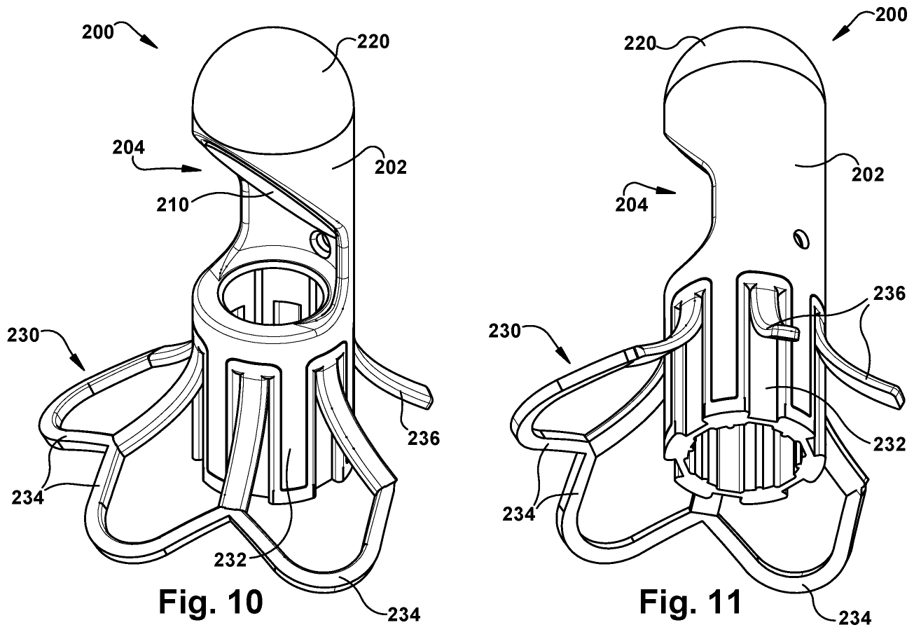

Another example configuration of the endcap 200 is illustrated in FIGS. 10-13. The endcap 200 can be similar or identical in configuration to the endcap illustrated in FIGS. 1-7. The endcap 200 implements a mirror 210 that can be similar or identical to the mirrors of FIGS. 8A-8C or FIGS. 9A-9C. The endcap parameters can also be similar or identical to those discussed above with those mirror configurations. The endcap 200 can therefore function similarly or identically to the endcap 100 in terms of visualizing the workspace and delivering the surgical tools 70 via the steerable sheath 50.

The endcap 200 is configured to include features in addition to those described above with regard to the endcap 100. The endcap 200 includes a body portion 202 that defines the side opening 204 and interior in which the mirror 210 is supported. The body portion 202 also includes the features (collar etc.) that facilitate connecting the endcap 200 to the endoscope 30. The body portion 202 can be formed of the same materials used to construct the endcap 150, such as a molded biocompatible polycarbonate material.

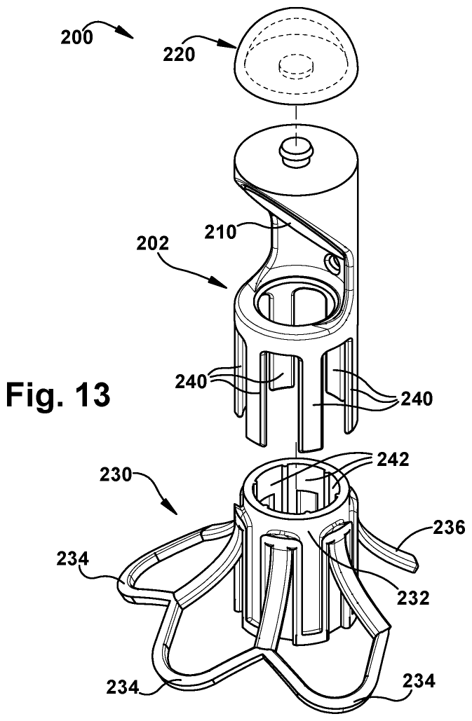

In addition to possessing the features described above in regard to the endcap 150, the endcap 200 includes a tip 220 and a basket 230. The tip 220 forms a domed end of the endcap 200 that is separate from, and connected to, the body portion 202. This is shown in FIG. 13. The connection between the tip 220 and the body portion 202 can, for example, be facilitated via a plug and socket type connection.

Advantageously, the tip 220, being separate form the body portion 202, can be formed of a material that is soft and flexible so as to aid in the navigation of the endoscope 30 with the endcap affixed thereto. The soft, flexible construction of the tip 220 can also help prevent tissue irritation or damage during delivery. The tip can, for example, be formed of a soft biocompatible material, such as a silicone rubber or a soft thermoplastic elastomer (TPE).

The basket 230 includes a central collar portion 232, a plurality of basket loops 234 that extend radially at an angle from the collar portion 232, and one or more fingers 236 that extend radially at an angle from the collar portion. The angles at which the loops 234 and fingers 236 extend are radially outward with respect to the collar 232 and away from the tip 220 of the endcap 200. The loops 234 and fingers 236 are spaced radially opposite each other on the collar portion 232. When connected to the body portion 202, the basket is axially aligned with the side opening 204 of the endcap 200.

The basket 230 is separate from, and connected to, the body portion 202. As shown in FIG. 13, this connection can be facilitated via a series of fingers 240, formed on the body portion 202, that are configured to be received by, and locked into, a series of slots 242 formed on an exterior of the collar portion 232.

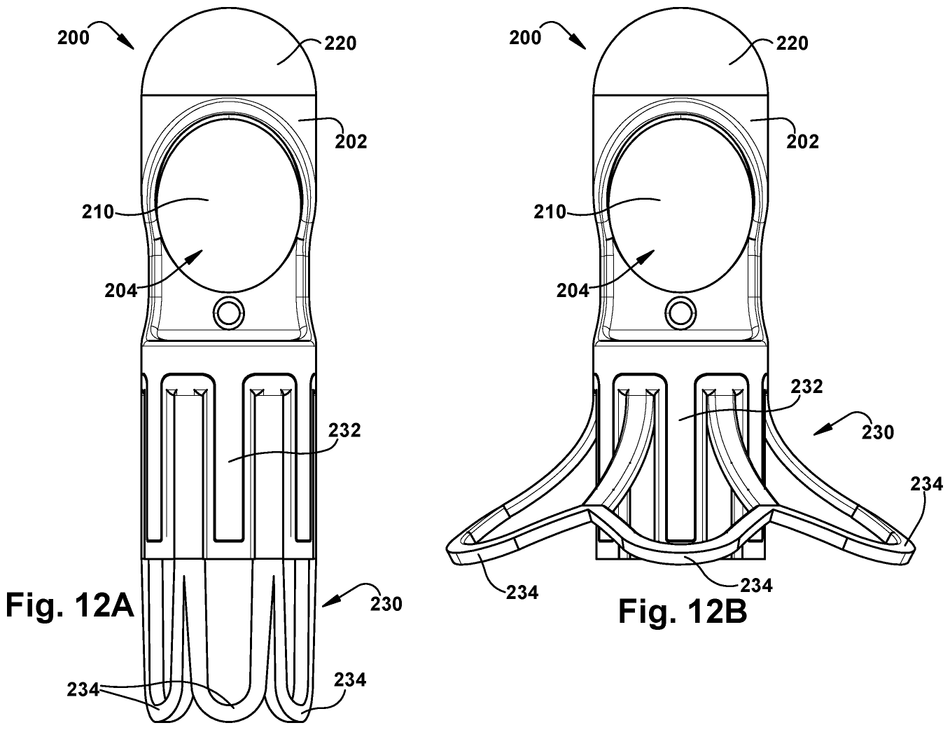

The basket 230 is formed of a flexible, resilient, biocompatible material, such as silicone rubber or TPE. the looped portion 234 and fingers 236 can thus be deflected relative to the collar portion 232 and the body portion 202 of the endcap 200. The looped portion 234 and fingers 236 can therefore be deflected from their normal positions, as shown in FIGS. 10, 11, and 12A, to their deflected positions, shown in FIG. 12B.

Advantageously, the basket 230 can help improve visualization of the workspace by supporting the endcap 200 at the worksite and also by expanding the tissues at the worksite. As the endoscope 30 and cap 200 are delivered to the workspace, the basket 230 deflects inward toward the collar 232 and body portion 202 as shown in FIG. 12B. This allows free, unrestricted navigation of the endoscope 230 without interference from the basket 230.

Once at the worksite, the endoscope 30 and cap 200 can be backed up, which allows the basket 230 to expand to the condition of FIG. 12A, with the looped portion 234 and/or fingers 236 engaging the surrounding tissue. The expanded basket 230, engaging the tissue, can help to expand the tissue and increase the FOV of the camera 34. At the same time, the expanded basket can support the end 32 of the endoscope 30 and cap 200 while the sheath 50 and tool 70 are maneuvered. This added support for the cap 200 and endoscope 30 can help prevent endoscope/cap movement in response to sheath 50 and/or tool 70 manipulation.

What have been described above are examples of the present invention. It is, of course, not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, modifications, and variations that fall within the spirit and scope of the appended claims.

We claim:

1. A system comprising an endcap configured to be fitted on a distal end of an endoscope having a forward-looking camera and a forward-facing working port, the endcap comprising a body configured to fit onto the distal end of the endoscope and a reflective surface supported by the body, the reflective surface being configured so that the forward-looking camera visualizes a workspace that is lateral of both the endcap and the distal end of the endoscope.

2. The system recited in claim 1, wherein the reflective surface is positioned in an interior of the body, the body further comprising a side opening through which the forward-looking camera visualizes the lateral workspace.

3. The system recited in claim 1, wherein the forward-looking camera is directed parallel to a central axis of the endoscope, wherein the reflective surface is angled with respect to the central axis so that the forward-looking camera views the lateral workspace via a reflection of the lateral workspace on the reflective surface.

4. The system recited in claim 1, further comprising a mirror that includes the reflective surface.

5. The system recited in claim 4, wherein the reflective surface is convex and increases the field-of-view (FOV) of the lateral workspace visualized by the camera.

6. The system recited in claim 5, wherein the convex reflective surface is spherical.

7. The system recited in claim 5, wherein the mirror has a length and a width, the length being greater than the width, wherein the convex reflective surface is aspherical, the radius of the convex reflective surface being larger across the length of the mirror, the radius of the convex reflective surface being smaller across the width of the mirror.

8. The system recited in claim 5, wherein the reflective surface comprises a flattened central portion surrounded by spherical or aspherical edge portions.

9. The system recited in claim 1, further comprising a steerable sheath configured to extend through the working port and exit the distal end of the endoscope, the steerable sheath having an inner lumen through which one or more tools can extend.

10. The system recited in claim 9, wherein the steerable sheath comprises a flexible tube having a steerable tip, the tip being actuatable to form a bend that enables the sheath to be steered outside the endcap in the lateral workspace.

11. The system recited in claim 10 wherein the endcap further comprises a redirecting surface configured to receive the steerable sheath and redirect the steerable sheath outside the endcap into the lateral workspace.

12. The system recited in claim 11, wherein the redirecting surface comprises a portion of the reflective surface.

13. The system recited in claim 11, wherein the redirecting surface is configured to enforce the lateral direction at which the steerable sheath exits the endcap into the lateral workspace.

14. The system recited in claim 10, wherein the steerable sheath comprises a concentric tube structure comprising nested concentric tubes, wherein actuation of the steerable tip is effectuated through applying an axial push-pull force to the concentric tubes.

15. The system recited in claim 14, wherein the concentric tubes are configured to have asymmetric elasticity along the portions of the tubes that extend along the steerable tip.

16. The system recited in claim 15, wherein the asymmetric elasticity of the tubes is created by cutout sections spaced lengthwise along the portions of the tubes that extend along the steerable tip.

17. The system recited in claim 9, wherein the tools comprise tools for performing an endoscopic retrograde cholangiopancreatography (ERCP) procedure.

18. The system recited in claim 9, wherein the tools comprise at least one of guidewires, cannulas, sphincterotomes, and baskets.

19. The system recited in claim 9, further comprising an endoscope having a forward-looking camera and a forward-facing working port, wherein the endcap is fitted onto the endoscope and the steerable sheath passes through the working port into the interior of the endcap, wherein the endcap is configured to deflect the steerable sheath toward a side opening of the endcap into the lateral workspace, and the steerable sheath is configured to be steered to a position and orientation in the lateral workspace, under the visualization afforded by the camera via the reflective surface, to allow a tool to be advanced through the steerable sheath into the lateral workspace.

20. The system recited in claim 19, wherein the endoscope comprises an illumination source and the reflective surface is configured to reflect light from the illumination source into the lateral workspace to illuminate the lateral workspace.

21. The system recited in claim 19, wherein the endoscope comprises a fluid delivery channel and the endcap is configured so that fluids delivered via the fluid delivery channel wash the reflective surface.

22. The system recited in claim 1, wherein the endcap body comprises a single piece of injection molded plastic configured to receive and support the reflective surface.

23. The system recited in claim 1, wherein the endcap body is free from moving parts.

24. The system recited in claim 1, wherein the endcap body comprises a collar configured to be press-fitted onto the distal end of the endoscope tube.

25. The system recited in claim 1, wherein the body of the endcap comprises a tip portion comprising flexible members configured to deflect in response to engaging tissue in order to facilitate navigation of the endoscope with the tip affixed thereto.

26. The system recited in claim 1, wherein the endcap further comprises a tip comprising a separate component connectable with the body, wherein the tip has a domed configuration and is constructed of a material that is soft and flexible, the tip being configured to deflect in response to engaging tissue in order to facilitate navigation of the endoscope with the tip affixed thereto.

27. The system recited in claim 1, wherein the endcap further comprises a basket, the basket comprising a plurality of loops and/or fingers that extend radially from the endcap body, wherein the loops and/or fingers are constructed of a material that is flexible and resilient so that the loops deflect toward the endcap body while the endoscope is advanced through tissue, the loops and/or fingers being configured to engage tissue and to deflect outward of the endcap body to space tissue from the endcap and to support the endcap and distal end of the endoscope in the workspace.

28. An endcap configured to be fitted on a distal end of an endoscope having a forward-looking camera and a forward-facing working port, the endcap comprising:
a body configured to fit onto the distal end of the endoscope, the body comprising a side opening providing access from an interior of the endcap to a lateral workspace exterior of the endcap, the workspace being lateral to both the endcap and the distal end of the endoscope;
a mirror supported in the interior of the endcap, the mirror comprising a reflective mirror surface in the field-of-view (FOV) of the forward-facing camera, the mirror surface being configured to place the lateral workspace within the FOV of the forward facing camera.
